(19) United States
(12) Patent Application Publication
(10) Pub. No.: US 2006/0069063 A1
(43) Pub. Date: Mar. 30, 2006

(54) Crosslinked polygalacturonic acid used for postsurgical tissue adhesion prevention

(76) Inventors: Yng-Jiin Wang, Taipei City (TW); Ming-Wei Lee, Taipei City (TW); Jia-Lu Hung, Sinjhuang City (TW)

Correspondence Address: BIRCH STEWART KOLASCH & BIRCH, PO BOX 747, FALLS CHURCH, VA, 22040-0747, US

(21) Appl. No.: 10/949,369

(22) Filed: Sep. 27, 2004

(51) Int. Cl. A61K 31/715; C08B 37/00

(52) U.S. Cl. 514/54; 536/123

(57) ABSTRACT

Disclosed is a crosslinked PGA that is obtained by crosslinking PGA polymer with a crosslinking reagent to develop a three-dimensional crosslinked PGA structure. The crosslinked PGA has good tissue antiadhesion and biocompatibility.

### BACKGROUND OF THE INVENTION

1. Field of the Invention

The present invention relates to a postsurgical tissue antiadhesion biomedical material, and in particular relates to a postsurgical tissue antiadhesion biomedical material manufactured by polygalacturonic acid (hereinafter referred to as PGA).

2. Prior Arts

Postsurgical tissue adhesion is one of the most urgent problems to be overcome for surgical works. Surgical adhesion can lead to small bowel obstruction (49-74%), infertility (15-20%), or chronic abdominal pelvic pain (20-50%), and thus increases postsurgical health care expense. The estimated cost for removing adhesion is about $NT twelve hundred million annually.

Undamaged peritoneal mesothelial cells contain fibrin solute, which is called tissue plasminogen activator (tPA). When tissues are injured during surgery, the injured tissues do not just decrease tPA but also increase plasminogen activator inhibitor; thus inhibit fibrin dissolution.

Wounds in abdomen cavity inhibit fibrin dissolution activity, and adhesion thus begins to develop. In the first three days, adhesion is caused by many materials forming fibrin matrix, which is gradually substituted by granulation tissue. In the forth day, most fibrin has disappeared and a great amount of fibroblast and collagen show up. In the fifth to tenth day, fibroblasts arrange into bundles and collagens slowly arise and deposit in the wound. After 1-2 months, collagen fiber tissues have formed and are not easily decomposed, thus leading to adhesion formation. In fact, adhesion formation is because of imbalance between fibrin deposit and its break down system.

When mesothelial cells are under repairment in the injured peritoneum, adhesion usually occurs at the same time. In general, adhesion formation takes approximately seven days. If a separation film is used to isolate injured tissue during the period, it can prevent adhesion from happening. As comparing to drugs, the film can be more precisely applied in injured areas and has fewer side effects.

Traditional approaches to reduce tissue adhesion include: (1) minimizing peritoneal trauma during surgery, (2) reducing inflammatory response, (3) inhibiting coagulation, (4) promoting fibrinolysis, and (5) placing a physical membrane to separate the injured and the normal tissues. Among these approaches, the most commonly used and most effective method is to place a physical membrane or film to separate the injured and the normal tissues. A polymeric film is the most commonly used one of the known physical films for antiadhesion.

Traditionally, an ideal adhesion prevention product shall be bioabsorbable, non-allergic-reactive, easy to apply, and capable of being fixed in position. Currently, in the United States, only three products have been approved for reducing postsurgical adhesion following intra-abdominal surgery. They are Interceed™ (Gynecare), Seprafilm™ (Genzyme), and Intergel™ (Lifecore). Interceed™ and Seprafilm™ are thin films, and Intergel™ is a hydrogel form. All the products are not fully satisfactory for clinical practice. This is due to their deficiencies in antiadhesion, biocompatibility, and bio-decomposability.

PGA is nature polysaccharides. Esterified PGA is the principle component of pectin that exists in plants and plays an important role of plant's nutrient diffusion process. PGA is a linear polymer, abundant in carboxyl and hydroxyl functional groups, consisting of repeated molecule (C₆ H₈ C₆)ₙ with α-1,4-glycosidic linkages with average molecular weight ranging from 50,000 and 150,000. Study of the molecular dynamics indicates that PGA chains form a 3-D network with both intra- and inter-molecular hydrogen bonding and with weak structure strength.

Pectin and PGA are extensively used in foodstuff industry and as a source for preparation of gel forming, ion exchanging, dye binding, and chelating materials. Nevertheless, PGA is rarely investigated for biomedical application except for the uses of drug delivery study.

This invention is aimed to develop a PGA based antiadhesion biomedical material in providing with good antiadhesion capability, biocompatibility, and bio-degradability.

### SUMMARY OF THE INVENTION

An object of the present invention is to provide a crosslinked PGA, which has a good postsurgical antiadhesion efficiency and thus can be used as an adhesion preventional biomedical material.

Another object of the present invention is to provide a corsslinked PGA having good biocompatibility.

In order to achieve and correspond to the above-mentioned objects, the present invention provides a crosslinked PGA that is accomplished by crosslinking PGA polymer with a crosslinking agent which crosslinks the polymer chains. Therefore, a 3-D network structure is obtained and has postsurgical adhesion prevention effect.

As it is indicated in the present invention, the antiadhesion capability of crosslinked PGA shown in the in vitro cell test is approximately similar to Seprafilm™, the most effective antiadhesion product. As further proceeded in cytotoxicity test, it designates that the crosslinked PGA does not have any cytotoxicity.

In addition, when the crosslinked PGA is implanted in vivo, its antiadhesion capability is better than Seprafilm™. Besides, the result of the histological examination demonstrates the crosslinked PGA implanted in animals does not elicit acute inflammatory reaction. This shows the crosslinked PGA has good biocompatibility. Moreover, the crosslinked PGA can be slowly decomposed as also shown in vivo test. Therefore, using the crosslinked PGA as an antiadhesion biomedical material in vivo can effectively prevent tissue adhesion and has no need of operation after the wound is healed.

The following embodiments will further illustrate the present invention but do not limit the mentioned scope. Those skilled in the art are able to perform and modify without departing from the scope of the invention.

### BRIEF DESCRIPTION OF THE DRAWINGS

The related drawings in connection with the detailed description of the present invention to be made later are described briefly as follows, in which:

FIG. 1 shows a scheme of a chemical reaction of PGA polymers crosslinked with carbodiimide as a crosslinking agent to form crosslinked PGA.

FIG. 2 shows a scheme of a chemical reaction of PGA crosslinked with photosensitive cinanmoyl compound as a crosslinking agent to form crosslinked PGA.

FIG. 3 shows gel contents of EDC crosslinked PGA immersed in saline for different days.

FIG. 4 shows gel contents of crosslinked PGA with different contents of cinnamyl bromide.

FIG. 5 shows MTT assays for adhesion and growth of fibroblast cells to antiadhesion films (n=4, p<0.5).

FIG. 6 shows photomicrographs of L929 fibroblast cell observed under phase-contrast microscope (100×). A1, A2, and A3 are the respective results of control group cultured for 3, 12, and 24 hours; B1, B2, and B3 are the respective results of Seprafilm™ group cultured for 3, 12, and 24 hours; and C1, C2, and C3 are the respective results of crosslinked PGA group cultured for 3, 12, and 24 hours.

FIG. 7 shows photopictures of injured tissue adhesion of rats for day 7 after surgery. A and B represent the control group and experimental group, respectively.

FIG. 8 shows histological photomicrographs of the wound sites stained with hematoxylin-eosin. A1 and A2 are the photomicrographs for day 7 and 14 of control group; and B1 and B2 are the photomicrographs for day 7 and 14 of experimental group.

FIG. 9 shows the neutrophil (A) and monocyte (B) numbers in peritoneal fluid in the 3 tested groups.

FIG. 10 shows cytotoxicity of PGA film and Seprafilm™.

### DETAILED DESCRIPTION OF THE PREFERRED EMBODIMENT

According to the present invention, the crosslinked PGA is obtained by crosslinking the polymer chains of PGA polymer with a crosslinking agent, which produces a biomedical material with postsurgical antiadhesion capability. Preferably, the crosslink degree of the crosslinked PGA is 60-100%, more preferably 70-95%, and most preferably 80-95%. Crosslinking agents can be used in this invention include but not limit to, carbodiimide compounds and photosensitive cinanmoyl compounds. An example of carbodiimide compounds includes 1-ethyl-3-(3-dimethylaminopropyl) carbodiimide (EDC), but not limit to it. Photosensitive compounds are compounds whose chemical property change after exposed to light or radioactivity. Advantages of using photosensitive compounds as crosslinking agents include: (1) conducting a light-crosslink reaction at low temperature or room temperature without initiator, (2) improving mechanical property of polymers with crosslinked structure after a light-crosslink reaction, (3) proceeding a light-crosslink reaction developing dimers and not releasing toxic materials, (4) crosslinking with UV light can achieve sterilized effect. Cinnamyl bromide is an example of photosensitive cinanmoyl compounds, but not limit to it.

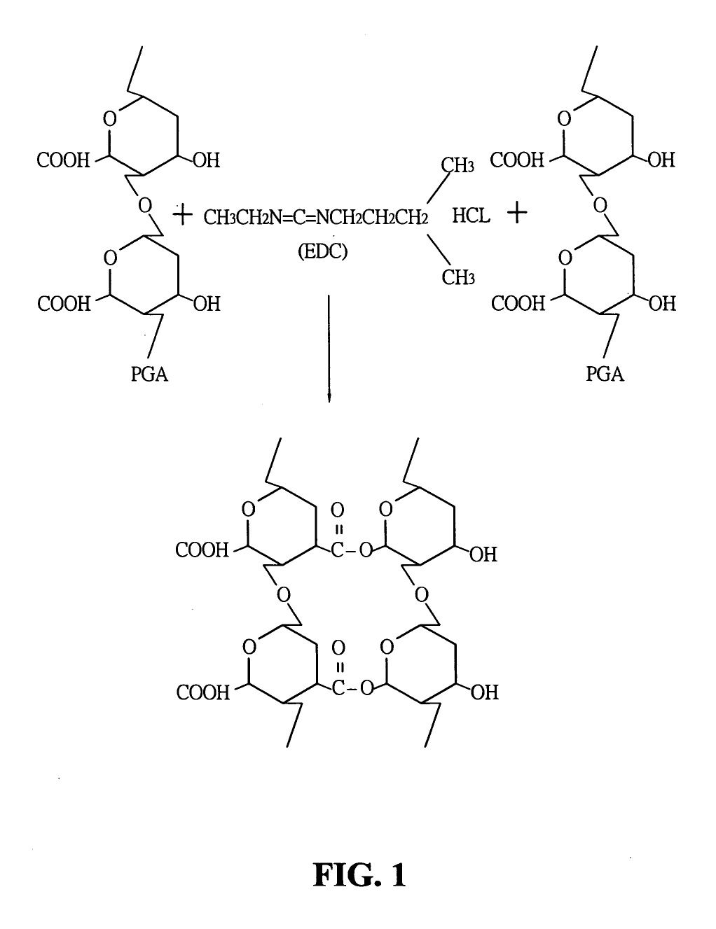

FIG. 1 is a scheme showing a chemical reaction of PGA polymers crosslinked with carbodiimide as a crosslinking agent to form crosslinked PGA. As it is shown in the scheme, carbodiimide crosslinks with the carboxyl and the hydroxyl groups of the PGA molecule side chain. Finally, an ester bond is formed between the two molecular chains, which is further developed into a three-dimensional network chemical structure unit shown in (I).

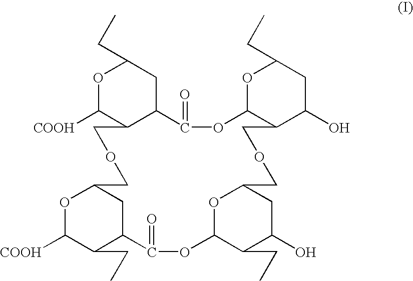

Crosslinked PGA is obtained by pouring PGA solution into a container and allowing it to evaporate and dry in order to form a film. The film is then in carbodiimide solution where crosslinking reaction is proceeded to form the crosslinked PGA as shown in formula (I). Preferably, the concentration of carbodiimide solution is 5-60 mM, more perferably 10-0 mM. Furthermore, preferably, the crosslink reaction time is 12-48 hours and more preferably 18-32 hours.

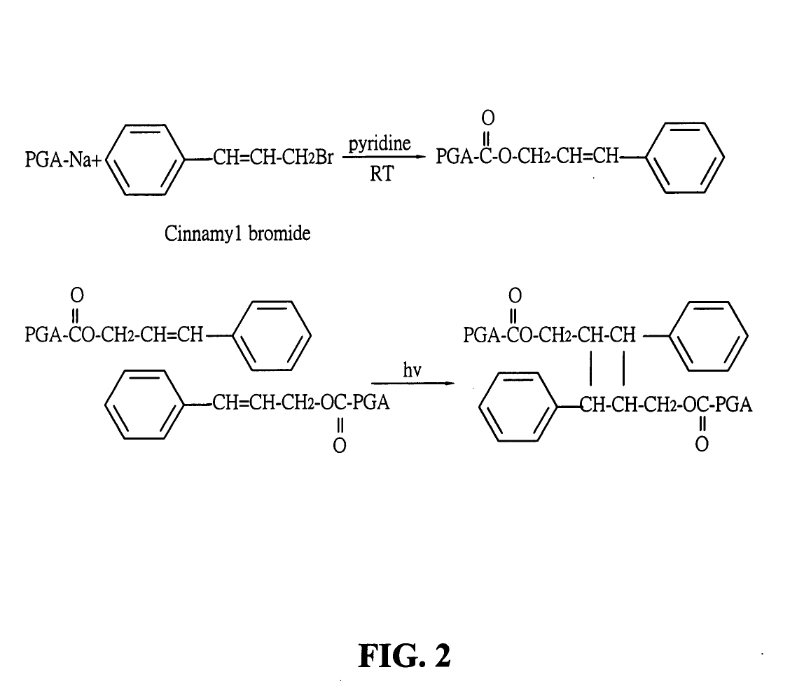

FIG. 2 is a scheme showing a chemical reaction of PGA crosslinked with photosensitive cinanmoyl compounds as a crosslinking agent to form crosslinked PGA. As it is shown in the scheme, photosensitive cinanmoyl compounds react with PGA polymer first, which makes the photosensitive cinanmoyl compounds to react with hydroxyl group of the PGA polymer chain to form chemical bonds. Then the photosensitive cinanmoyl modified PGA polymer chains crosslink to each other to form a cyclobutane crosslinking structure by UV radiation. UV radiation initiates the crosslink reaction and crosslinks modified PGA. The product with three-dimensional network structure is obtained, and as indicated in (II).

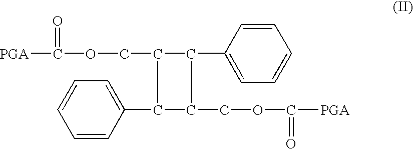

To prepare the crosslinked photosensitive PGA with various grafting ratios, the molar ratio of photosensitive cinanmoyl compound and carboxyl group of PGA is adjustable (for example, 0.5-1.5:1). Then the photosensitive PGA is crosslinked by UV radiation.

Currently, Seprafilm™ from Genzyme Corporation is considered the best tissue antiadhesion material. This film is coupled and crosslinked by EDC with the same molar concentration of hyaluronic acid (HA) and carboxymethylcellulose (CMC). Although Seprafilm™ can inhibit adsorption and proliferation of fibroblast, it still has deficiencies, including fast decomposition rate within the body, high brittleness and easy to break, completely gelification as wet, not easy to use clinically, and high cost. Therefore, to affirm the characteristics, such as tissue antiadhesion and biocompatibility of crosslinked PGA, Seprafilm™ is taken as a comparative material to evaluate the postsurgical tissue antiadhesion effect of the present invention.

In order to examine the tissue antiadhesion effect of the crosslinked PGA of the present invention, tests in vitro and in vivo are proceeded respectively. Among these, fibroblast is used in vitro test with 3-(4,5-dimethylthiazol-2-yl)-2,5 diphenyltetrazolium bromide (herein after is referred to as MTT) assay to analyze the cell's adhesion to the tested object. The result shows that the crosslinked PGA and Seprafilm™ have similar fibroblast adhesion. But as further observed under light microscope of fibroblast morphology, fibroblast on the crosslinked PGA has a lower growth rate than Seprafilm™. Thus, the crosslinked PGA has better antiadhesion effect than Seprafilm™.

In vivo test, crosslinked PGA and Seprafilm™ are respectively implanted in rats for tissue adhesion examination. When implanted in rat, crosslinked PGA exhibits most promising antiadhesion potential with only 1 out of 21 rats. This antiadhesion potency is significantly higher than that of 11 out of 21 operated rats for Seprafilm™. Thus, the result suggests that the crosslinked PGA has better tissue antiadhesion effect than Seprafilm™. In addition, histological study of near implanted area reveals that the crosslinked PGA does not induce any inflammatory reaction. Thus the crosslinked PGA has good biocompatibility. Moreover, the results show that the crosslinked PGA does not display cytotoxicity.

Summing up, the crosslinked PGA of the present invention has better postsurgical tissue antiadhesion effect, biocompatibility, and anti-biological decomposition than Seprafilm™. Moreover, the corsslinked PGA also had Seprafilm™ characteristics. Therefore, the crosslinked PGA is a preferred tissue antiadhesion material used in postsurgery than Seprafilm™.

Having been briefly described, the present invention will be further explained with examples and figures illustrating its practice set forth below. These examples and figures should not, however, be considered to limit the scope of the invention, which is defined by the appended claims.

### EXAMPLE 1

### Preparation of Crosslinked PGA Film with Carbodiimide

Sodium polygalacturonate is dissolved in deioized water with pH adjusted to 6.4 to obtain a 2% (w/v) aqueous solution. About 25 mL of PGA solution is poured into a glass dish (diameter 10 cm) and evaporated at 45° C., 1 atm for 3 days until the weight of film remains constant.

The resulted PGA film is crosslinked by immersing in a solution containing 15 mM EDC, 80% ethanol and 20% water for 24 hours at room temperature. Then, the crosslinked film is washed with 95% ethanol three times to remove the unreacted residues. Finally, it is dried at room temperature to obtain the crosslinked PGA film.

The gel contents of resulted PGA reveal the crosslinked degree of gels, and can be applied to determine a gel degraded rate. The dry crosslinked PGA is cut into a size of 2 cm×2 cm and weighed to obtain a weight W₁. The films are immersed and swelled in saline at 37° C. for 1-3 days respectively. Then the wet films are dried at 60° C. for 12 hours and weighed to obtain a weight W₂. The gel content of crosslinked PGA is calculated by the following equation:
Gel content (%)=(W₂/W₁)×100%

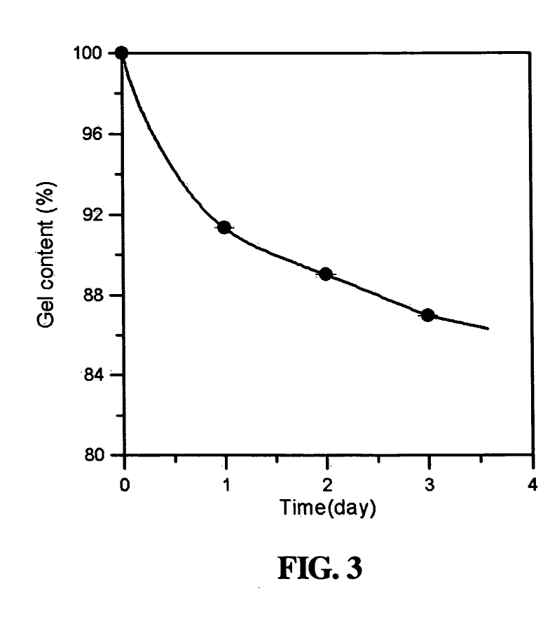

FIG. 3 shows the gel content of EDC crosslinked PGA immersed in saline for different days. It shows that the gel contents are 91%, 89%, and 86% respectively for 1, 2, and 3 days. This demonstrates that the degraded rate of the crosslinked PGA in vitro is slow. Moreover, it reveals the crosslinked degree of the gel is between 95-80% from the gel content analysis.

### EXAMPLE 2

### Preparation of Crosslinked PGA Film with Photosensitive Cinanmoyl Compound

Three preparations of crosslinked PGA film containing different photosensitive cinanmoyl group are performed. In each preparation, 1 g of sodium polygalacturonate is dissolved in 80 mL deioized water with pH adjusted to 6.4. 20 mL of dimethyl sulfoxide (DMSO) is added and mixed well. Then, cinnamyl bromide is added into each mixed solution with a molar ratio of 0.5, 1, and 1.5 to PGA (0.454 g, 0.908 g, and 1.362 g respectively), pyridine is also added with the same molar ratio of cinnamyl bromide (0.185 mL, 0.371 mL, and 0.835 mL respectively). The solutions are then set in room temperature for 4 days.

When the above-mentioned reaction is completed, four times volume of alcohol is added and the solution is centrifuged at 10,000 rpm for 60 minutes. Precipitate from centrifugation is dissolved in a small volume of deionized water and stirred for one day to be dissolved completely. Then add four times volume of alcohol and repeat the above-mentioned purification process twice. The final product dissolved in deionized water is freeze-dried.

Respectively dissolve 0.2 g, 0.4 g, and 0.6 g of synthesized PGA-cinnamyl bromide in 20 mL deionized water to obtain 1% w/v, 2% w/v, and 3% w/v solution. The solutions are centrifuged after complete dissolution. The supernatant is poured into 10 cm×10 cm film containers and placed in oven at 45° C. to dry. Films are then developed.

The crosslinked PGA film is cut into 2 cm'2 cm and placed in oven to dry. A weight of W₁ is obtained by weighing the film until the weight remains constant. Then the film is immersed in dimethylformamide (DMF) for a day and exposed to UV radiation for an hour to crosslink. After crosslinking, the solution is displaced by 100% ethanol to remove DMF residues, then the gel is in deionized water for one minute. A weight of W₂ is obtained by weighing the dried film immersed in water.
Gel content=(W₂/W₁)×100%

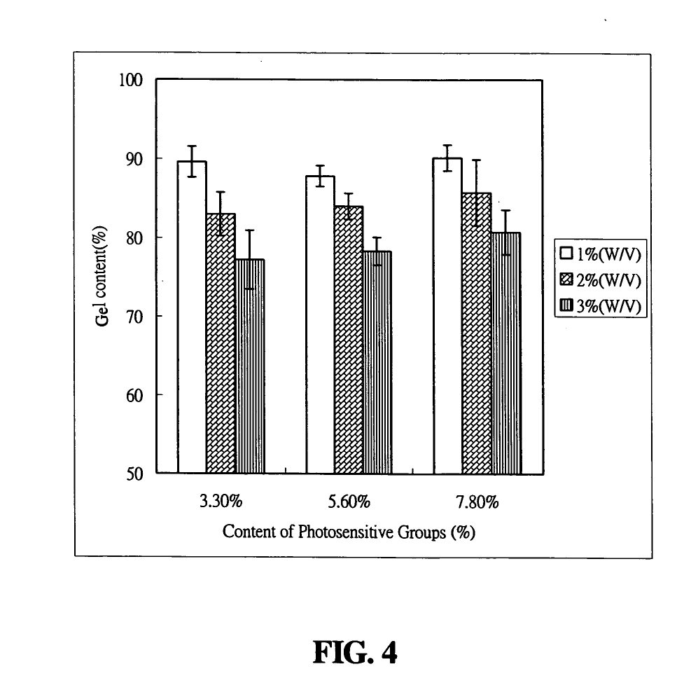

FIG. 4 shows gel contents of crosslinked PGA with different contents of cinnamyl bromide. It suggests that the more photosensitive groups exist, the higher gel contents contained in the films. Thus the crosslinked PGA film can be obtained at higher crosslink degree. In addition, the gel content decreases with the thickness of films. It is presumable that the increasing width of gels may result in a decreasing photosensitivity in the photocrosslinking reaction.

Therefore, these results reveal that the gel contents of the crosslinked PGA films increase with the photosensitive groups in crosslinked PGA, and the crosslinked PGA films with higher crosslink degree are achievable. Moreover, the crosslink degree of the films apparently decreases with the film thickness.

### EXAMPLE 3

### Anti-Cell Adhesion In Vitro

For determining cell adhesion and growth on the antiadhesion films, the crosslinked PGA films obtained in Example 1 are placed in the bottom of each well of a 48-well tissue culture plate (NUNC, Roskilde, Demark). Fibroblast (L-929) cells are added to the plate with 3×10⁴ cells each well and incubated at 37° C.

Seprafilm™ are placed in the bottom of each well of a 48-well tissueculture plate and 3×10⁴ fibroblast cells (L-929) are added to each well and incubated at 37° C. For comparison, each well of a 48-well tissue culture plate is added with 3×10⁴ fibroblast cells (L-929) without any antiadhesion film and incubated at 37° C. as a control group.

After culturing of fibroblast cells for 12, 24, 48, or 72 hours, MTT assay is carried out to determine the adhesion of cells to anti-cell adhesion films by the absorbance at 570 nm.

For experimental data of each group, four independent measurements are conducted. Differences between the control group and the experimental groups are analyzed statistically by using two-samples t-test. The differences observed between samples are considered significantly for P values lower than 0.05.

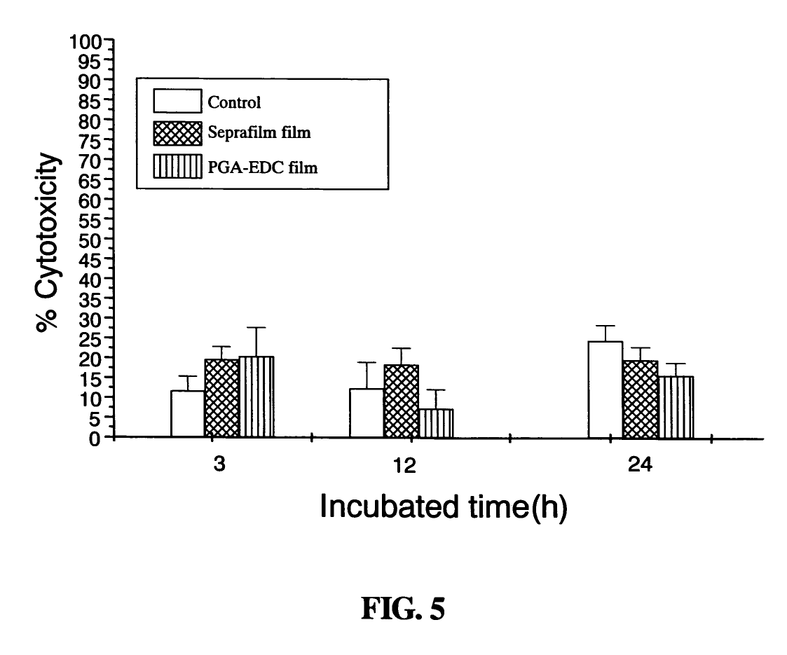

FIG. 5 shows MTT assays for adhesion and growth of fibroblast cells to antiadhesion films. In the first twelve hour cultures, the absorbances of crosslinked PGA film and Seprafilm™ are 0.3 and 0.4 respectively, and there is no significant difference between the two groups. Comparing to the absorbances of control group (the value of 1.1), crosslinked PGA film and Seprafilm™ show only less than 30% of fibroblast adhesion. Nevertheless, from the slopes of absorbance increasing with incubation time (dotted line in FIG. 5), it is presumable that once the fibroblasts adhere to the tested films, the cell growth rates are about the same. This indicates that both films have ability of anti-cell adhesion and exhibit no toxicity for cells.

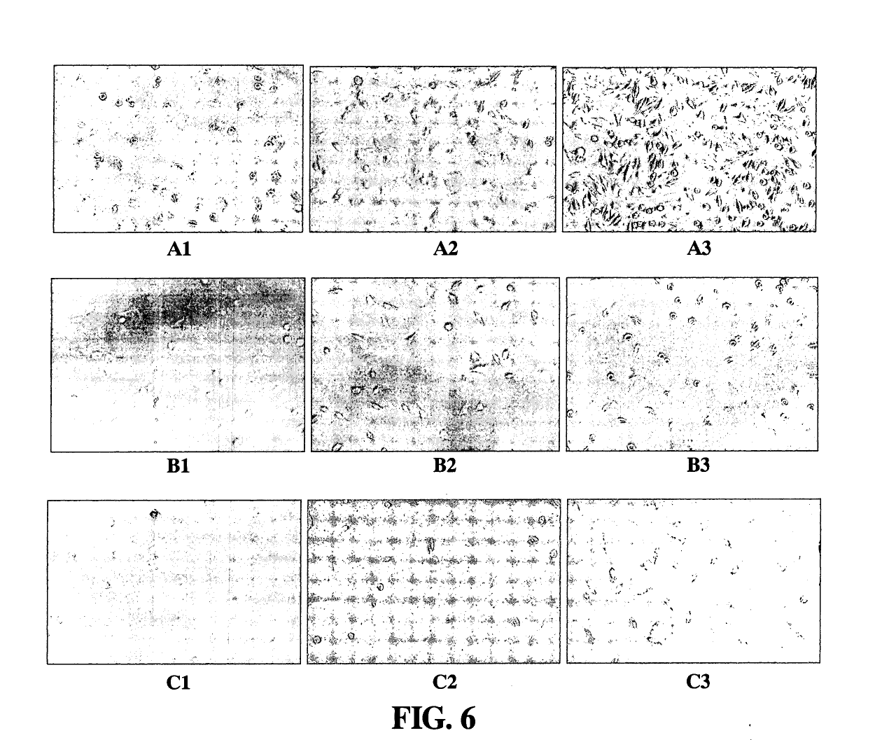

From the above-mentioned results of MTT assays, it shows that the effect of anti-cell adhesion films depends on the early stage of cell culturing. FIG. 6 shows the morphology of L929 fibroblast cell culturing in a 48-well tissue culture plate within the first day, which morphology is observed under phase-contrast microscope. The number of cells adhered to both of Seprafilm™ and crosslinked PGA films are apparently much less than those to the control group. Furthermore, cells on crosslinked PGA film seem to take longer time (24 hours) to flatten and elongate than those on Seprafilm™. From this and the result showing a lower cellular vitality on crosslinked PGA film as compared with Seprafilm™ in FIG. 5, suggest that crosslinked PGA film is more effective to prevent tissue adhesion when implanted in vivo.

### EXAMPLE 4

### Anti-Cell Adhesion In Vivo

A total of 63 Sprague-Dawley rats weighted 200-250 g are anesthetized with 4% trichloroacetaldehyde monohydrate (1 mL/100 g) to conduct aseptic midline laparotomy. Incise a 3-5 cm wound in the middle of abdomen and utilize an expander to push open the wound. Make a size of 1 cm×1 cm wound in abdominal cavity wall and bleed the intestinal wall capillary corresponding to the wound using a surgical knife. Fasten the two ends of the intestine in abdominal cavity wall with sutures. Crosslinked PGA film and Seprafilm™ film are implanted between the wound in abdominal cavity wall and intestinal wall as an experimental and comparative group respectively. None of antiadhesion film is implanted in rats in a control group.

Then, the rats are sacrificed respectively on day 3, 7, and 14 after surgery to examine the process of adhesion formation at the injured site. The abdominal wall of the injured sites are removed and fixed in 10% formalin solution. The tissues are processed by the standard procedure for histological examinations and the tissue sections are examined with hematoxylin-eosin stain.

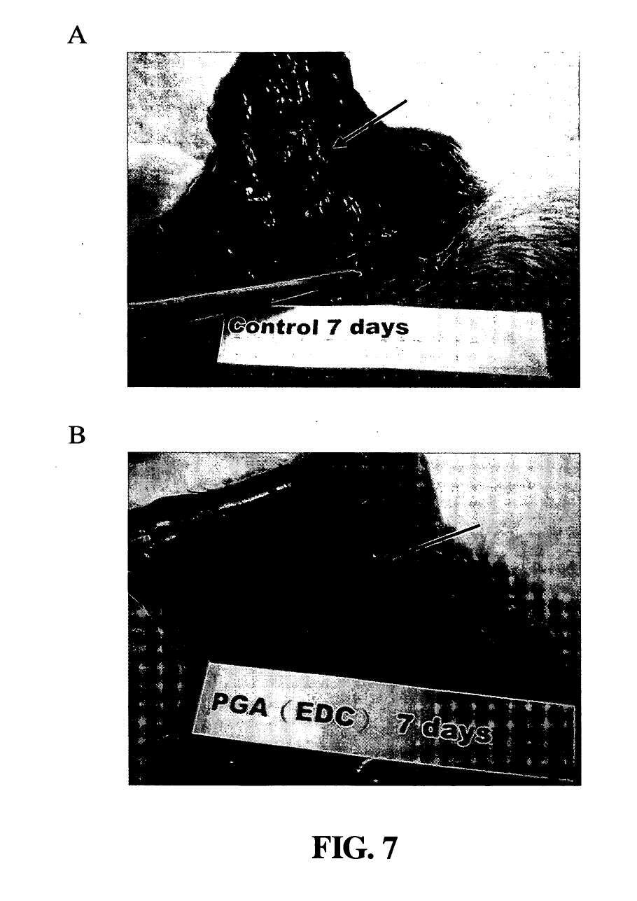

The occurrence of tissue adhesion between the caecum and the peritoneal wall is examined on day 3, 7 and 14 after surgery. Rats in control group without any film implantation show adhesion formation in 6 out of 7 on day 7 after surgery (FIG. 7A). On the other hand, on day 7 after surgery, the adhesion formation ratios in comparative and experimental group are lower than in control group. Rats show adhesion formation in 4 out of 7, 0 out of 7 and 2 out of 7 in comparative group treated with Seprafilm™ films and in experimental group treated with crosslinked PGA films prepared according to Example 1 (Exp 1 group) and 2 (Exp 2 group), respectively (FIG. 7B). On day 14 after surgery, rats show adhesion formation in 5 out of 7 in control group but 4/7, 1/7 and 1/7 in comparative, Exp 1 and Exp 2 group respectively. From all test results (Table 1), numbers of rats having adhesion formation on day 3, 7 and 14 are summed up to calculate the total adhesion formation ratio in the experimental period. From Table 1, in total 21 rats each group, rats with adhesion formation are 18 and 11 in control and comparative group, but 1 and 4 in Exp 1 and Exp 2 group, respectively. Thus, it suggests that the crosslinked PGA has good anti-adhesion ability, and even superior to Seprafilm™ film in preventing postsurgical tissue adhesion

TABLE 1
Postsurgical Tissue Adhesion Formation of Rats Operated

Days after surgery

| | | | | Data |
|---|---|---|---|---|
| Group | Day 3 | Day 7 | Day 14 | summed up |
| Control | 7/7 | 6/7 | 5/7 | 18/21 |
| Comparative | 3/7 | 4/7 | 4/7 | 11/21 |
| Exp 1 | 0/7 | 0/7 | 1/7 | 1/21 |
| Exp 2 | 1/7 | 2/7 | 1/7 | 4/21 |

All data listed in Table 1 are shown as numbers of rats with adhesion/numbers of rats operated.

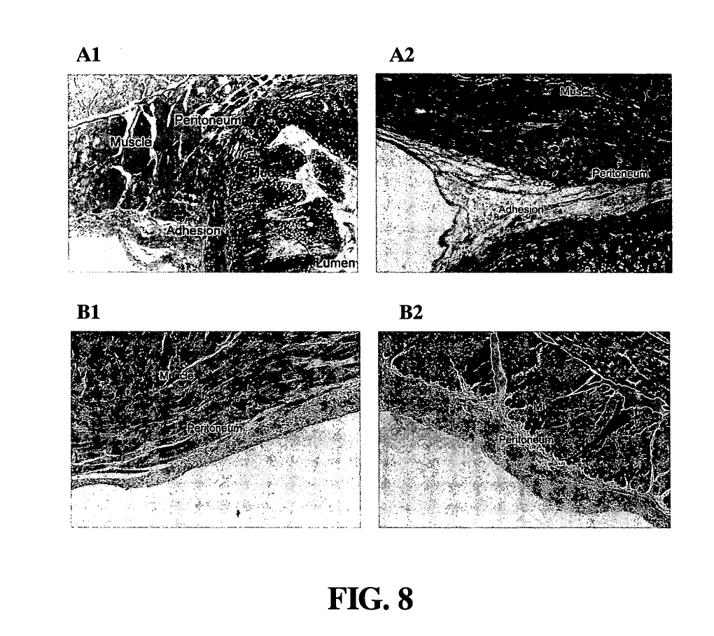

FIG. 8 shows histological photomicrographs of the wound site stained with hematoxylin-eosin. In control group, adhesion tissue with thick layer of fibroblast cells and collagen fiber between caeum and the peritoneal wall can be observed clearly on day 7 (A1) and day 14 (A2) after surgery. Moreover, the numbers of neutrophil and monocyte are higher on day 7 than on day 14 after surgery, and fibroblasts infiltrate to the adhesion area.

Exp 1 group shows that epithelialization of the peritoneal wall inner surface is completed without adhesion formation FIG. 8 (B1 and B2). Although the inflammatory cells are prominent, the cell numbers decrease significantly on day 14. In addition, the mesothelial layer of peritoneal wall is not edematous and the tissue healing continues from day 7 to 14 after surgery. Thus, histological analysis reveals that the crosslinked PGA films do not induce any specific inflammatory reaction, or cause a low fibrotic response as compared with the control group.

Determination of leukocyte population in peritoneal fluid provides information of the degree of inflammatory response in surrounding an implant. This data provide more informations to explain the results of histological studies. In this study, hemocytometer is used to determine the numbers of peritoneal fluid neutrophils and monocytes elicited by crosslinked PGA and Seprafilm™ films.

The rats operated according to the above-mentioned method are sacrificed on day 3, 7 and 14 after surgery. For peritoneal fluid collection, about 2 mL of the Dulbecco's Modified Eagle's Medium (DMEM) with added heparin is injected into the peritoneal cavity. The peritoneal fluid is aspirated through pipette with a bulb tip. The amounts of neutrophils and monocytes in the collected fluid are determined by using a standard clinical hemocytometer (ADVIA 120, Bayer).

In the early period after injury (up to 3 days), the most pronounced leukocytes in healing of peritoneal lesions are neutrophils. It derives from acute inflammatory reaction and neutrophils are involved in the pathophysiology of intraperitoneal adhesion formation. The population changes of the peritoneal fluid neutrophils for the three tested groups are shown in IG. 9A. For all tested groups, the numbers of neutrophils reach maximum within the first 3 days after surgery and then gradually decreas over the 14 days period of observation. In the PGA-treated animals, the number of neutrophils slightly increases but still within the control levels (P value 0.096) on day 14 after surgery. The analyzed results of neutrophils in peritoneal fluid indicates that PGA film and Seprafilm™ do not elicit any acute inflammatory reaction as compared to the control group.

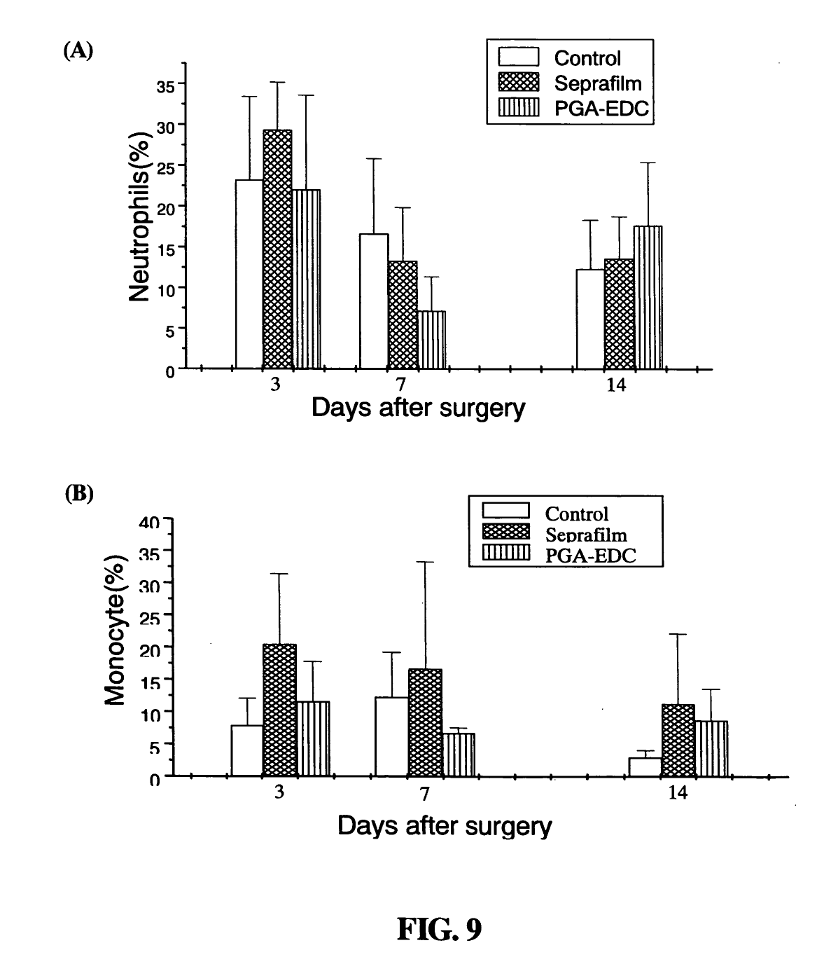

In addition, the numbers of peritoneal fluid monocytes are also determined to evaluate whether the degraded antiadhesion materials have elicited inflammatory reaction. An ideal surgical films for adhesion prevention is biodegradable and is not reactive to tissue. Monocytes are attracted to the site of a foreign material as a result of chemotactic signals pertaining to the inflammatory process. The numbers of peritoneal fluid monocytes in the three tested groups are shown in FIG. 9B. More monocytes are found in the case of Separfilm™-treated rats as compared with that of the control group after surgery shown in the figure (P value 0.013, 0.026, and 0.038 for day 3, 7, and 14). The numbers of monocytes found in crosslinked PGA-treated rats are not statistically different from that found in the control group (P value 0.275 and 0.218 for day 3 and 7). However, on day 14 after surgery, crosslinked PGA-treated group experienced higher number of monocytes as compared with the control group (P value 0.005). These results can be interpreted as that Seprafilm™ is rapidly biodegraded in the early period after injury and elicits inflammatory reaction. On the other hand, the PGA film is degraded slower and only elicits slightly inflammatory reaction at a later stage.

### EXAMPLE 5

### Cytotoxicity Test

The cytotoxicity of the antiadhesion film is determined from the levels of LDH (lactate dehydrogenase) released by the cells incubated with the polymeric films under investigation. LDH is a stable cytoplasmic enzyme present in all cells and rapidly released into the cell culture supernatant upon damage of the plasma membrane. The release of LDH from cells is determined by using a LDH-Cytotoxicity Asaay Kit (BioVision). The procedure is as follows.

First, crosslinked PGA films and Seprafilm™ films are separately immersed in the cell culture medium in 48-well tissue-culture plates. Aliquots of 400 μl cell suspensions (L-929 fibroblast, NCTC clone 929) are added to each well at a density of 4×10⁴ cells/mL and incubated at 37° C. for 3, 12, and 24 hours.

After a predetermined incubation period, the medium is aspirated and centrifuged at 250 g for 10 minutes. The supernatant (100μl ) is taken from each well and transferred into 96-well plate. The BioVision kit reagent (100 μl) is then added to each well and incubated for 30 minutes at room temperature. The absorbance of the reaction mixture at 490-500 nm is measured using a microtiter plate reader at wavelength of 600 nm.

Positive control (complete release of LDH) is performed using the cell lysate of 1% Triton X-100, which is known to completely rupture cell membranes. For background release, cells are plated onto 48-well tissue-culture plates and the cell lysate is analyzed as described above.

The cytotoxicity (%) of the samples is calculated by the following equation. Each experimental value represents an average value of four experiments repeated.
Cytotoxicity (%)=[(tested sample−background release)/(positive control−background release)]×100%

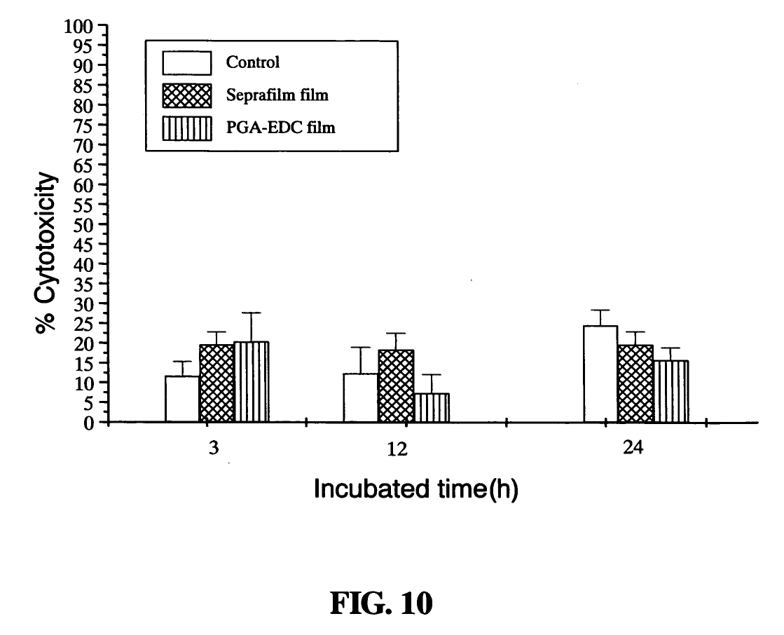

FIG. 10 illustraes cytotoxicity of crosslinked PGA film and Seprafilm™ film. As shown, the cytotoxicity indices are all less than 20% for both films after 3, 12, and 24 hours of direct contact of L-929 fibroblast cells of crosslinked PGA film and Seprafilm™ film. When compared with the control group, there is no statistical difference of the release of LDH, and it indicates that both crosslinked PGA film and Seprafilm™ film are nontoxic to the cells.

Summing up, the crosslinked PGA is superior to Seprafilm™ in preventing tissue adhesion in vivo. It may be presumable that the structure strength of the crosslinked PGA is stronger than Seprafilm™, and it may be biodegraded more easily due to the weak structure strength of Seprafilm™. Before the tissue is completely healed, Seprafilm™ may have been already biodegraded and leads to adhesion in injured site. Furthermore, as shown in FIG. 7, the crosslinked PGA is able to remain its structure and to be isolated during the healing period thus do not cause tissue adhesion. In addition, once Seprafilm™ expands in water, its surface turns into wet and sticky that is inconvenient for users to operate. This does not occur in the crosslinked PGA and thus easy to be operated in implanting in vivo. Moreover, the main ingredient of Seprafilm™ is hyaluronic acid (HA), which is very expensive as compared to PGA, which is the main ingredient of crosslinked PGA, can be prepared from pectin mentioned previously. Thus, its production will cost less. After all, the crosslinked PGA is superior to Seprafilm™ for postsurgical tissue antiadhesion.

Furthermore, two kinds of compounds have mentioned, carbodiimide and cinnamyl bromide, are used as crosslinking agents to react with PGA in order to prepare crosslinked PGA, all with superior postsurgical tissue adhesion prevention. Therefore, it can be logically reasoned that those skilled in the art can easily prepare the crosslinked PGA with different crosslinking agents. That is, no matter what crosslinking agent is used and as long as it crosslinks with PGA to obtain its crosslinked product, the efficacy shall be the same or similar to the present invention.

What is claimed is:

1. A crosslinked PGA used for postsurgical tissue adhesion prevention, which is obtained from a crosslinking reaction of PGA polymer with a crosslinking agent, wherein crosslinking degree of the crosslinked PGA is 50-100%.

2. The crosslinked PGA according to claim 1, wherein the crosslinking agents is selected from the group consisting of carbodiimide compound and photosensitive cinanmoyl compound.

3. The crosslinked PGA according to claim 2, wherein the carbodiimide compound is 1-ethyl-3-(3-dimethylaminopropyl) carbodiimide (EDC).

4. The crosslinked PGA according to claim 3, wherein the crosslinked PGA comprises a chemical unit with a chemical formula (I) as follows:

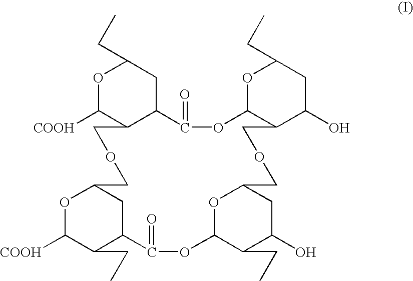

5. The crosslinked PGA according to claim 2, wherein the photosensitive cinanmoyl compound is cinnamyl bromide.

6. The crosslinked PGA according to claim 5, wherein the crosslinked PGA comprises a chemical unit with a chemical formula (II) as follows:

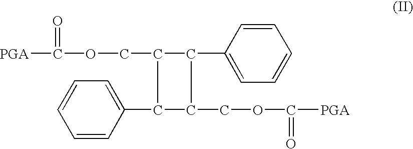

7. The crosslinked PGA according to claim 1, wherein the crosslinked PGA is not cytotoxic.

8. The crosslinked PGA according to claim 1, wherein the crosslinked PGA is biocompatibile.

9. A method for preparing crosslinked PGA used for postsurgical tissue adhesion prevention, comprising the steps of:
(A) preparing a PGA solution;
(B) preparing a crosslinking agent solution; and
(C) producing crosslinked PGA by a crosslinking reaction of the PGA solution with the crosslinking agent solution, wherein, crosslinking degree of the crosslinked PGA is 50-100%.

10. The method according to claim 9, wherein the crosslinking agent is a carbodiimide compound.

11. The method according to claim 10, wherein the carbodiimide compound is 1-ethyl-3-(3-dimethlyaminopropyl) carbodiimide (EDC).

12. The method according to claim 10, wherein step (A) further comprises a step of drying crosslinked PGA solution to obtain PGA films.

13. The method according to claim 12, wherein step (C) comprises a step of immersing the PGA films into the crosslinking agent solution to perform the crosslinking reaction.

14. The method according to claim 13, wherein the concentration of the crosslinking agents solution is 5-60 mM and the reaction time is 12-48 hours.

15. The method according to claim 11, wherein the crosslinked PGA comprises a chemical unit with a chemical formula (I) as follows:

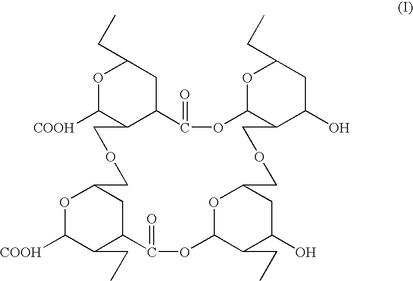

16. The method according to claim 9, wherein the crosslinking agent is photosensitive cinanmoyl compound.

17. The method according to claim 16, wherein the photosensitive cinanmoyl compound is cinnamyl bromide.

18. The method according to claim 16, wherein in step (C), a molar ratio of the crosslinking agent to the PGA is 0.5-1.5:1.

19. The method according to claim 16, wherein step (C) comprises a step of exposing to UV light.

20. The method according to claim 17, wherein the crosslinked PGA comprises a chemical unit with a chemical formula (II) as follows: [chemical structure]
